(19) United States
(12) Patent Application Publication
(10) Pub. No.: US 2018/0235526 A1
(43) Pub. Date: Aug. 23, 2018

(54) WEARABLE OXIMETER FOR RESPIRATION MEASUREMENT AND COMPENSATION

(71) Applicant: Nonin Medical, Inc., Plymouth, MN (US)

(72) Inventors: Bryant Austin Jones, St. Louis Park, MN (US); Eric Danielson, Plymouth, MN (US); Gregory J. Rausch, Minnetonka, MN (US); Marcus A. Kramer, Circle Pines, MN (US)

(21) Appl. No.: 15/752,188

(22) PCT Filed: Aug. 12, 2016
(86) PCT No.: PCT/US16/46878

§ 371 Date: Feb. 12, 2018

Related U.S. Application Data
(60) Provisional application No. 62/205,211, filed on Aug. 14, 2015.

(51) Int. Cl. A61B 5/1455; A61B 5/113; A61B 5/00

(52) U.S. Cl. CPC A61B 5/14552; A61B 5/113; A61B 5/6833; A61B 5/6815; A61B 5/721; A61B 5/6826; A61B 5/6843

(57) ABSTRACT

Systems, devices, and methods for measuring one or more parameters at a tissue site of a patient are disclosed. The system can include a sensor that can be configured to measure a first physiological parameter. The physiological parameter can be associated with oxygenation of arterial blood. A second physiological parameter can be associated with respiration of the patient. The system can include a processor that can be configured to use the first and second physiological parameters to determine a waveform associated with the oxygenation of the arterial blood, and the processor can be configured to compensate for a signal artifact associated with motion of the tissue site.

### CLAIM OF PRIORITY

This application claims the benefit of U.S. Provisional Application No. 62/205,211, filed Aug. 14, 2015, which is hereby incorporated by reference herein in its entirety.

### BACKGROUND

Currently available oximetry technologies are designed to function on relatively easy to use sites which exhibit strong pulsatile signals, such as the fingers, head and ears. Other tissue sites, such as the chest, may provide an advantage but skin characteristics and signal artifacts impair the accuracy of oximetry measurement.

### OVERVIEW

An example of the present subject matter includes a wearable oximeter for use on the chest. Among other things, movement of the chest due to breathing presents a particularly difficult signal environment for oximetry. Current approaches developed for legacy sites are inadequate to deal with the problems caused by respiratory motion.

A device can be configured to measure one or more physiological parameters of a patient. A patient can wear the device, for example, on a digit (such as a finger or a toe), a limb (such as a calf or forearm), or the torso (such as the chest). In an example, a device may also be coupled to the patient at a tissue site, such as at the chest (e.g., on the pectoral muscle), the back, an arm, a leg, the head (e.g., on the frontal cortex), or the torso, for example. In an example, the device can be bonded to the tissue site by an adhesive. The adhesive can include a resilient pad in which one side of the pad can be adhered to the skin of the patient and the device can be adhered to another side of the adhesive pad. In one example, the device can be coupled to a location on the patient using the adhesive pad. The adhesive pad can be shaped or sized to fit a particular tissue site.

Motion can be associated with the tissue site, and thus a device affixed to the site. In an example, patient respiration (breathing) can be a source of motion. Motion can also be attributed to physical activity, turbulence from a moving vehicle, or if the patient changes posture, such as sitting down or standing up. In an example, motion of the tissue site can be caused by an external source, such as clothing in contact with the device and relative motion between the device and the patient's skin. Another example of a source of motion of the tissue site can be attributed to device inertia relative to cessation of patient movement. There can be other sources of motion. The adhesive pad can be configured to adhere to the skin of the patient at a particular tissue site during such motion.

The present subject matter includes systems, devices, and methods as described herein. For example, the present subject matter can include structure to stabilize a bond between the device and patient tissue. In one example, the present subject matter provides a consistent interface for the coupling between the device and the skin. This improved consistency can improve accuracy and repeatability of the measurements provided by the device.

In one example, the device can include a first sensor having an optical emitter and a detector. The emitter can be configured to transmit selected wavelengths of light directed at a tissue site and the detector can receive the resulting light as it emerges from the tissue site or light reflected from the tissue site. A signal from the detector can be analyzed to determine a measure of oxygenation based on light attenuation. One example of a device can be used for non-invasive measurement of arterial oxygenation saturation. The device can include a processor configured to operate at least one sensor. The device can include a first sensor configured to measure oxygenation and include a second sensor. The first sensor and the second sensor can provide respective output signals to the processor. In one example, the second sensor is configured to measure a parameter corresponding to motion of the device (or motion of the tissue site). In one example, the second sensor includes a force sensor. A force sensor can be configured to measure a force between the device and the skin of the patient.

In an example, the present subject matter can be configured to compensate for motion associated with the tissue site or compensate for various skin types. Skin type information can be used to improve the accuracy of the measurement. The output from the device can include a physiological waveform.

In one example, an optical sensor uses light passing through a tissue bed in an adhesive patch to determine a measurement of respiration rate.

In one example, a secondary sensor (such as a pressure sensor, an optical energy sensor, or a physical strain sensor) is coupled to the tissue using an adhesive patch and the secondary sensor provides a respiration rate measurement.

In one example, a motion artifact is cancelled or mitigated using a signal from a secondary sensor coupled to tissue using an adhesive patch. The motion artifact can be associated with respiration or physical motion (voluntary or involuntary).

In one example, a motion artifact is cancelled or mitigated by a mechanical apparatus that maintains a relatively constant force between the tissue and the sensor. The mechanical apparatus can be viewed as a constant force unit (CFU) and can be secured to the tissue with an adhesive patch.

This Overview is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates a schematic of a device according to one example.

FIG. 2 illustrates a diagram of a device according to one example.

FIG. 3 illustrates a diagram of a device according to one example.

FIG. 4 illustrates a block diagram of a device according to one example.

FIG. 5 illustrates a diagram of a device according to one example.

FIG. 6 illustrates a flow chart of a method according to one example.

FIG. 7 illustrates a system according to one example.

### DETAILED DESCRIPTION

The amount of physical force (or pressure) applied at a sensor-to-skin interface of a measurement device will affect the ability of the device to accurately measure SpO₂, heart rate and respiration rate. Variations in the applied pressure due to varying skin characteristics, breathing, and motion can cause undesirable variations in these readings.

An example of the present subject matter includes a method to improve the stability and consistency of the skin-sensor interface and can improve the repeatability, consistency and overall accuracy of the system. In addition, patient comfort can be improved by reducing the surface area of the adhesive patch by which the device is affixed to a tissue site. Various approaches are described herein.

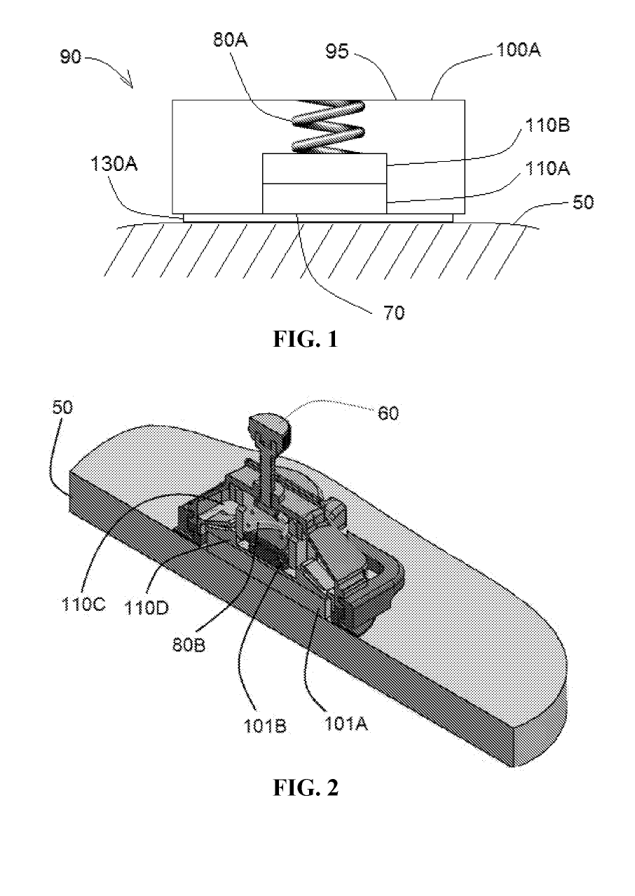

FIG. 1 illustrates system 90 including device 100A affixed to tissue 50. Device 100A includes housing 95 that carries both first sensor 110A and second sensor 110B. Housing 95 includes contact surface 70. In the example shown, contact surface 70 is affixed to tissue 50 by adhesive 130A. Adhesive 130A can include a glue or other bonding agent and in various examples, includes a pad having adhesive on opposing faces.

First sensor 110A is affixed within housing 95 such that the contact surface 70 is aligned with a detector element of first sensor 110A. First sensor 110A can include one or more optical emitters and one or more optical detectors. First sensor 110A can provide an output signal corresponding to arterial oxygenation (pulse oximetry) or venous oxygenation (tissue oximetry or regional oximetry), or a measure of other optical-based parameter.

Second sensor 110B is affixed within housing 95 such that it provides a measure of a force, pressure, or acceleration as to device 100A. In the example shown, second sensor 110B is coupled to housing 95 by elastic element 80A, here illustrated as a helical spring.

FIG. 2 illustrates an example of a device. In the figure, the device includes a housing fabricated of first component 110C and second component 110D. In one example, first component 110C has a female attribute and second component 110D has a male attribute. First component 110C is affixed to tissue 50 and the second sensor 101B is affixed to an interior surface of second component 110D. Second component 110D is configured to slide within a feature of first component 110C in a direction perpendicular to a plane of tissue 50, thereby allowing physical forces between the two to equilibrate. In this configuration, elastic element 80B provides a pre-load (force or pressure). Elastic element 80B exerts a bias force on second sensor 101B. In this example, second sensor 101B includes a force sensor. Second sensor 101B can be configured to provide periodic or continuous measurements of the force (pressure) applied between the device and the tissue. Low friction sliding elements can be provided to reduce the effects of friction (or stiction) and to allow the sensor to float gently on the tissue.

First sensor 101A, in the example illustrated, includes a pulse oximetry sensor. Adjuster 60 includes a knob on a threaded shaft and allows tuning of the bias force exerted on second sensor 101B.

In the example illustrated, male and female components are configured to cooperatively couple and a spring is utilized to preload a force onto the skin. A force sensor is carried within the housing and low friction sliding elements allow relative movement within the housing.

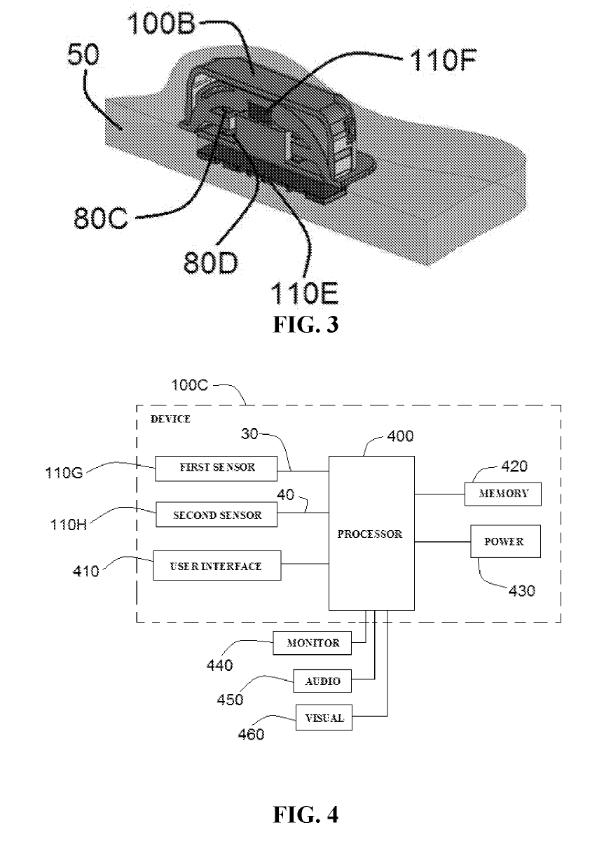

FIG. 3 illustrates a diagram of device 100B according to one example. Device 100B is shown affixed to tissue 50. Device 100B includes first sensor 110E and second sensor 110F. First sensor 110E can includes a pulse oximetry sensor and second sensor 110F can include a force sensor. Elastic element 80C and elastic element 80D correspond to a first flexure component and a second flexure component, here each of which are illustrated in the form of a leaf spring. Alignment and placement of elastic element 80C and elastic element 80D allows for relative motion of the first sensor 110E with respect to second sensor 110F.

The dual flexure elements in this example allow the sensor to float perpendicular to the tissue surface, and thus equalize the force (pressure) of the sensor upon the skin. This configuration can reduce or eliminate the effects of friction in the mechanism. This example also includes a force sensor configured to provide periodic or constant data acquisition of the force (pressure) applied.

FIG. 4 illustrates a block diagram of device 100C according to one example. Device 100C includes processor 400 coupled to first sensor 110G and second sensor 110H. An output signal from first sensor 110G is conveyed to processor 400 by link 30 and an output signal from second sensor 110H is conveyed to processor 400 by link 40. Processor 400, in the example illustrated, accesses executable instructions and data in memory 420. Power 430 provides electric energy to processor 400 and other components of device 100C. User interface 410 provides data input and data output for the benefit of a user and can include a wired or wireless display and an input device such as a keyboard or user operable switch. In addition, processor 400 is coupled to monitor 440 which provides storage and control functions. Processor 400 is also wired or wirelessly coupled to audio interface 450 and visual interface 460, providing audible and visual data, respectively, to a user. Device 100C may also be configured as a sealed unit for use in an environment such as underwater, at high altitude, or in a vacuum.

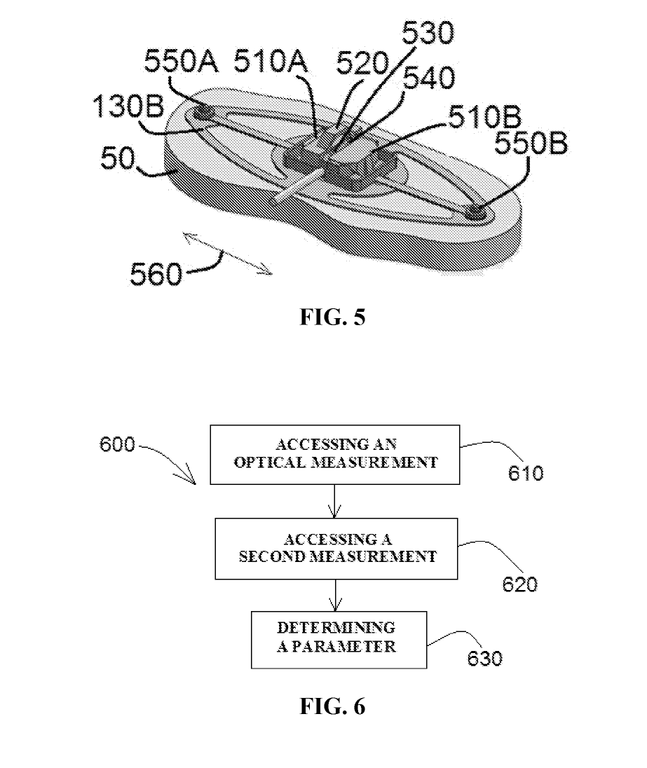

FIG. 5 illustrates a diagram of a device according to one example. In the example illustrated, the device includes anchor 550A and anchor 550B. Anchors 550A and 550B provide attachment points by which adhesive 130B is coupled to a tissue 50 and coupled to the sensor structure. Adhesive 130B is configured in a skeletal manner as shown and allows movement of the sensor structure in the directions indicated by the arrowheads on line 560. Movement of the sensor structure in a lateral direction is detected by a sliding aperture 510A and sliding aperture 510B. Mirror 520 includes a pair of reflective elements arranged at an angle to provide an indication of sliding motion. In one example, mirror 520 includes a pair of mirrors disposed at an angle of 45 degrees. The combination of mirror 520 and sliding aperture 510A and sliding aperture 510B provides an indication as to relative movement of the sensor structure. In one example, emitter 530 and detector 540 are configured to provide a respiration waveform. In one example, another optical sensor (including an emitter and detector) provides an oximetry signal at the tissue site.

In this example, a respiration artifact can be mitigated or removed from the SpO₂ waveform, using sensor affixed using a suspension apparatus. The optical elements may be independent of, or integrated into, the suspension apparatus. The suspension apparatus provides a signal corresponding to lateral displacement across the patch surface. The example illustrated is configured to provide a waveform to allow extraction of a SpO₂ measurement without the respiratory artifact, or to provide a respiration waveform alone, or in conjunction with a sensor to measure another physiological metric. In addition, the configuration illustrated allows for measuring respiration through the optics disposed on the device.

FIG. 6 illustrates a flow chart of method 600 according to one example. Method 600 includes, at 610, accessing an optical measurement. The optical measurement can be derived from a sensor including an optical emitter and an optical detector. The optical measurement can correspond to oxygenation of blood or tissue. At 620, method 600 includes accessing a second measurement. The second measurement can be derived from the optical elements associated with the optical measurement or be derived from another sensor. The signal for the second measurement can be provided by a displacement sensor, a force sensor, an accelerometer, or other sensor. At 630, method 600 includes determining a parameter. In one example, the parameter is associated with oxygenation compensated for a movement artifact.

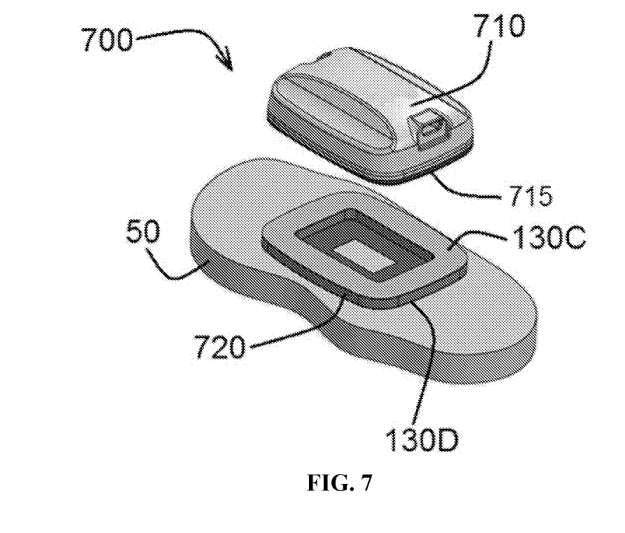

FIG. 7 illustrates system 700 according to one example. System 700 includes oximetry sensor 710. In various examples, sensor 710 is configured for pulse oximetry or tissue oximetry. Dock 715 provides a mounting structure for engagement of a corresponding attachment structure which allows attachment to tissue 50. Dock 715 can include a shaped component that securely engages with layer 720. Layer 720 can include a resilient membrane fabricated of foam or rubber or other elastomeric material or flexible material. Layer 720 is affixed to tissue 50 by adhesive 130D and is affixed to sensor 710 by adhesive 130C. In the example shown, layer 720 and adhesive 130C and adhesive 130D provides a double-sided low-trauma skin contact adhesive. In one example dock 715 includes a forward mounted configuration, wherein the sensor and electronics are easily attached and removed from the adhesive patch.

### ADDITIONAL NOTES

The examples illustrated can be combined in various arrangements to provide an assortment of configurations.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described.

However, the present inventors also contemplate examples in which only those elements shown or described are provided.

Each of these non-limiting examples can stand on its own, or can be combined in various permutations or combination with one or more of the other examples.

Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, an apparatus, system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A device comprising:
a housing having a bond surface, the bond surface configured to couple the housing to a tissue site;
a first sensor coupled to the housing, the first sensor having a light emitter and a light detector, the first sensor configured to provide a first signal corresponding to a first parameter, the first parameter corresponding to blood chemistry of arterial blood at the tissue site, and wherein the first sensor has a sensor surface configured to contact the tissue site; and
a second sensor coupled to the housing, the second sensor configured to measure a second signal corresponding to a second parameter, the second parameter associated with motion of the housing.

2. The device of claim 1 wherein the second sensor includes a force sensor.

3. The device of claim 1 wherein the first parameter corresponds to oxygenation of the arterial blood and the second parameter corresponds to a respiration rate.

4. The device of claim 1 wherein the housing includes a first component and a second component, wherein the first component includes the bonding surface and wherein the second component is configured to movably couple to the first component.

5. The device of claim 1 wherein the first sensor is coupled to the housing by an elastic element.

6. The device of claim 1 wherein the second sensor is coupled to the housing by a slide member, and wherein the second signal corresponds to a displacement relative to the bond surface.

7. The device of claim 1 wherein the bond surface includes a flexible material.

8. A system comprising:
at least one sensor having a sense surface and configured to provide a first signal corresponding to tissue proximate the sense surface; and
a processor coupled to the at least one sensor, the processor configured to determine a first physiological parameter and a second physiological parameter based on the first signal, the first physiological parameter associated with oxygenation and the second physiological parameter associated with physical motion of the at least one sensor, wherein the first physiological parameter is determined using the second physiological parameter.

9. The system of claim 8 wherein the processor is configured to execute instructions to determine a waveform, determine a measure of pulse rate, determine a measure of pulse rate variability, determine a measure of pulse amplitude, amount of hemoglobin, or a level of dysfunctional hemoglobin.

10. The system of claim 8 wherein the at least one sensor includes a force sensor configured to provide a force signal corresponding to a force between the sense surface and the tissue.

11. The system of claim 8 wherein the at least one sensor includes an optical sensor configured to measure a displacement.

12. The system of claim 8 further comprising a housing wherein the housing includes a first component coupled to the sense surface.

13. The system of claim 12 wherein the housing includes a second component moveably coupled to the first component, the second component aligned substantially perpendicular to the sense surface.

14. The system of claim 8 further comprising a first elastic element coupled to the at least one sensor, the first elastic element configured to exert a force relative to the sense surface and an adjoining surface.

15. The system of claim 14 further comprising a second elastic element configured to enable motion of the at least one sensor relative to the housing.

16. A method comprising:
accessing an optical measurement associated with oxygenation of arterial blood at a tissue site;
accessing a force measurement associated with motion of the tissue site; and
determining a first physiological parameter associated with a waveform, the waveform determined using the optical measurement and the force measurement.

17. The method of claim 16 wherein determining the waveform includes deriving at least one of an arterial oxygenation of the tissue site, a pulse rate, respiration rate, regional tissue oxygenation, total hemoglobin content, or level of dysfunctional hemoglobin.

18. The method of claim 16 further comprising determining a second physiological parameter corresponding to the tissue site and wherein determining the first physiological parameter includes determining using the second physiological parameter.

19. The method of claim 16 wherein the force measurement corresponds to respiration and wherein determining the first physiological parameter includes compensating the optical measurement for the respiration.

20. The method of claim 16 wherein accessing the optical measurement includes affixing a sensor to the tissue site using an elastic element wherein the elastic element is configured to at least substantially maintain a position of a force sensor relative to the tissue site.
